(19) United States
(12) Patent Application Publication
(10) Pub. No.: US 2011/0038902 A1
(43) Pub. Date: Feb. 17, 2011

(54) ANTIPERSPIRANT COMPOSITIONS AND PRODUCTS AND METHODS FOR MANUFACTURING ANTIPERSPIRANT COMPOSITIONS AND PRODUCTS

(75) Inventors: Brittany Phipps, Peoria, AZ (US); Thomas Doering, Scottsdale, AZ (US)

Correspondence Address: THE DIAL CORPORATION, 19001 N. Scottsdale Road, SCOTTSDALE, AZ, 85255, US

(73) Assignee: The Dial Corporation, Scottsdale, AZ (US)

(21) Appl. No.: 12/729,974

(22) Filed: Mar. 23, 2010

Related U.S. Application Data
(63) Continuation-in-part of application No. 12/540,532, filed on Aug. 13, 2009.

(51) Int. Cl. A61K 8/894; A61Q 15/00; B28B 7/24

(52) U.S. Cl. 424/401; 424/65; 424/68; 264/255

(57) ABSTRACT

Antiperspirant products and methods for manufacturing antiperspirant products are provided herein. In accordance with an exemplary embodiment, an antiperspirant composition comprises a container and an antiperspirant product housed within the container. The antiperspirant product comprises a first portion and a second portion. The first portion comprises an emulsion having a water phase and an oil phase. The oil phase comprises cetyl PEG/PPG-10/1 dimethicone. The second portion has a composition different from the first portion.

### CROSS-REFERENCES TO RELATED APPLICATIONS

This is a continuation-in-part of application Ser. No. 12/540,532, filed Aug. 13, 2009, which is herein incorporated by reference in its entirety.

### FIELD OF THE INVENTION

The present invention generally relates to antiperspirant compositions and products and methods for manufacturing antiperspirant compositions and products, and more particularly relates to antiperspirant products that exhibit antiperspirant efficacy with the enhanced feel of deodorants, antiperspirant compositions comprising such antiperspirant products, and methods for manufacturing such antiperspirant compositions and products.

### BACKGROUND OF THE INVENTION

Antiperspirants and deodorants are popular personal care products used to prevent or eliminate sweat and body odor caused by sweat. Antiperspirants typically prevent the secretion of sweat by blocking or plugging sweat-secreting glands, such as those located at the underarms. Deodorants counteract or mask the unwanted odors caused by bacterial flora in secreted sweat.

Antiperspirant solid wax sticks are desired by a large majority of the population because of the presence of active antiperspirant compounds that block or prevent the secretion of sweat and its accompanying odors and because of their ease of application. A solid wax antiperspirant product is applied to the skin by swiping or rubbing the stick across the skin, typically of the underarm. However, antiperspirant users often are disappointed in the chalky, brittle, and/or crumbly application of the solid wax stick across the skin. Deodorants, on the other hand, typically provide a better “glide” and no caking when applied to the underarm skin. The term “glide” typically is used to denote the friction between the antiperspirant and/or deodorant product and the skin. The smoother the glide, or the less friction between the product and the skin, the more desirable the product is to users. While deodorants typically exhibit smoother glide than solid wax antiperspirant sticks, they do not prevent or eliminate the secretion of sweat as do antiperspirants because they do not contain active antiperspirant compounds. Active antiperspirant compounds generally cannot be added to deodorants because the alkalinity of the deodorants causes the antiperspirant compounds, typically acidic, to precipitate or settle out of the deodorants. Thus, there is a need for antiperspirant products that exhibit antiperspirant efficacy with the feel of deodorants.

Antiperspirant emulsion formulas that exhibit minimal crumbling or caking are known. Such products typically include an emulsion of an aqueous phase containing an antiperspirant salt and an oil phase containing, for example, a volatile silicone, fragrances, gellants, and other additives. While such formulas may provide the preferred glide of deodorants, they often have a wet or sticky feel. In addition, conventional antiperspirant emulsions do not exhibit the antiperspirant efficacy that solid wax antiperspirant sticks do.

Accordingly, it is desirable to provide antiperspirant products that exhibit enhanced antiperspirant efficacy with the smooth glide of deodorants. It is also desirable to provide antiperspirant products that do not exhibit a wet or sticky feel. In addition, it is desirable to provide methods for manufacturing such antiperspirant products. Furthermore, other desirable features and charkteristics of the present invention will become apparent from the subsequent detailed description of the invention and the appended claims, taken in conjunction with the accompanying drawings and this

### BRIEF SUMMARY OF THE INVENTION

Antiperspirant products and methods for manufacturing antiperspirant products are provided herein. In accordance with an exemplary embodiment, an antiperspirant composition comprises a container and an antiperspirant product housed within the container. The antiperspirant product comprises a first portion and a second portion. The first portion comprises an emulsion having a water phase and an oil phase. The oil phase comprises cetyl PEG/PPG-10/1 dimethicone. The second portion has a composition different from the first portion.

In accordance with another exemplary embodiment, an antiperspirant product comprises an outer portion comprising an emulsion having a water phase and an oil phase. The oil phase comprises cetyl PEG/PPG-10/1 dimethicone. An inner portion at least substantially bisects the outer portion. The inner portion has a composition different from the outer portion.

In accordance with a further exemplary embodiment, a method for manufacturing an antiperspirant product is provided. The method comprises depositing a first portion of the antiperspirant product into a mold. The first portion comprises an emulsion having a water phase and an oil phase. The oil phase comprises cetyl PEG/PPG-10/1 dimethicone. The first portion is allowed to at least partially solidify. A second portion of the antiperspirant product is deposited into the mold. The second portion has a composition different from the first portion. The second portion is allowed to at least partially solidify.

### BRIEF DESCRIPTION OF THE DRAWINGS

The present invention will hereinafter be described in conjunction with the following drawing figures, wherein like numerals denote like elements, and wherein:

FIG. 1 is a perspective view of an antiperspirant product in accordance with an exemplary embodiment;

FIG. 2 is a top view of the antiperspirant product of FIG. 1;

FIG. 3 is an exploded perspective view of an antiperspirant composition comprising the antiperspirant product of FIG. 1 in a container in accordance with an exemplary embodiment;

FIG. 4 is a perspective view showing the initial step of a filling assembly being inserted into a container in a process for manufacturing the antiperspirant composition of FIG. 3 in accordance with an exemplary embodiment;

FIG. 5 is a perspective view of an outer nozzle assembly of the filling apparatus shown in FIG. 4;

FIGS. 6 and 7 are cross-sectional views of the center nozzle tube shown in FIG. 5;

FIG. 8 is an enlarged detailed view of a portion of the outer nozzle assembly of FIG. 5;

FIG. 9 is a perspective view of an inner nozzle assembly of the filling apparatus shown in FIG. 4;

FIG. 10 is a perspective view of the inner nozzle assembly shown in FIG. 9, with one half of the nozzle housing removed to show the interior of the inner nozzle assembly;

FIG. 11 is an elevational view of the inner nozzle assembly shown in FIG. 10 and FIG. 12 is an exploded view showing internal components of the inner nozzle assembly;

FIG. 13 is a perspective view showing a further step in the manufacturing process of FIG. 4;

FIG. 14 is a cross-sectional view taken along line 14-14 in FIG. 13;

FIG. 15 is a perspective view showing a further step in the manufacturing process of FIGS. 4 and 13;

FIG. 16 is a cross-sectional view taken along line 16-16 in FIG. 15;

FIG. 17 is a perspective view showing a further step in the manufacturing process of FIGS. 4, 13, and 15;

FIG. 18 is a cross-sectional view taken along line 18-18 in FIG. 17;

FIG. 19 is a partially exploded perspective view of a portion of a nozzle assembly used in an alternative process for manufacturing the antiperspirant composition illustrated in FIG. 3 in accordance with an exemplary embodiment;

FIG. 20 is a fully exploded perspective view of the nozzle assembly of FIGS. 19; and

FIG. 21 is a cross sectional view showing the nozzle assembly of FIG. 19 filling a container.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to antiperspirant products that exhibit antiperspirant efficacy and that also provide a smooth glide when applied to the skin. The various embodiments of the antiperspirant products comprise a first portion and a second portion. The first portion is a solid emulsion that comprises cetyl PEG/PPG-10/1 dimethicone as an emulsifier. The use of cetyl PEG/PPG-10/1 dimethicone as an emulsifier in an antiperspirant product causes the antiperspirant product to exhibit skin feel characteristics that are typical of deodorant products. For example, with cetyl PEG/PPG-10/1 dimethicone, the antiperspirant products glide onto skin with less friction, that is, in a smoother and moister manner, than conventional antiperspirants. The second portion is an invisible dry, solid wax formula that provides enhanced antiperspirant efficacy, long-lasting fragrance, odor protection, bacteria control, and/or other desired purposes and/or functions. (Accordingly, the first portion, the second portion, or both portions of the antiperspirant product provides antiperspirant efficacy.) It has been unexpectedly found that when both portions are applied together, to the extent that the first portion, by itself, would ordinarily exhibit a wet and sticky feel, together the portions exhibit a dry, smooth glide without the caking or crumbling that the second portion would ordinarily exhibit by itself. These application aesthetics are not possible with any other conventional antiperspirant or deodorant product.

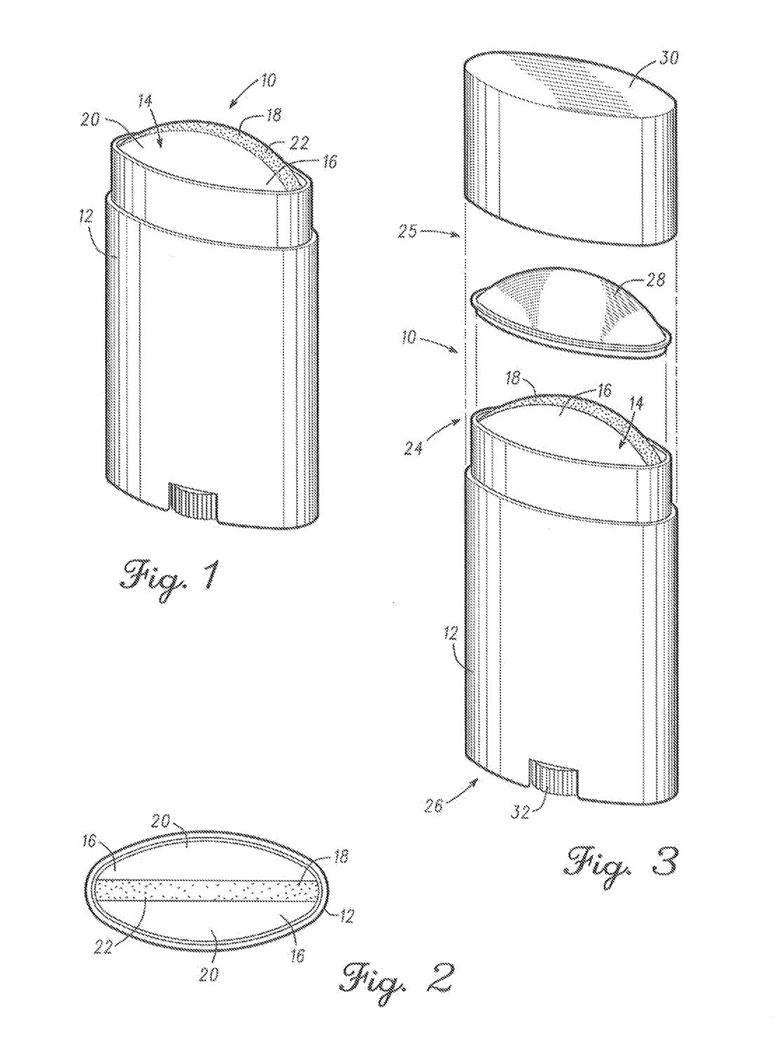

Referring to FIGS. 1 and 2, an antiperspirant product 10 in accordance with an exemplary embodiment comprises a first portion 16 and a second portion 18. The term “portion,” as used herein, includes the section or sections of the antiperspirant product having the same composition; for example, two sections having the same composition but separated by a third section (for example, a central stripe) having a different composition constitute a single “portion.” First portion 16 may have a color different from that of second portion 18 or the portions may be of the same color. Antiperspirant product 10 has an application surface 14 that is substantially dome-shaped and that is configured to be applied to skin, such as, for example, an underarm. Antiperspirant product 10 also may comprise a container or dispenser 12 for dispensing first portion 16 and second portion 18 to the skin.

In one exemplary embodiment, first portion 16 is an outer portion and second portion 18 is an inner portion and application surface 14 comprises a surface 20 of first portion 16 that is bisected by an adjacent surface 22 of second portion 18. First portion 16, being a water-and-oil emulsion formula as noted above, is positioned on outside regions of the antiperspirant product 10 while second portion 18, being a solid wax formula, is positioned between the regions of first portion 16. This configuration is a preferred configuration because the first portion has a melting point that is higher than the melting point of the second portion. During manufacture, as described in more detail below, the second portion is formed after the first portion and, thus, will not melt the already-prepared first portion. However, it will be appreciated that the invention is not limited to the configuration of first portion 16 and second portion 18 illustrated in FIGS. 1 and 2. Rather, it will be appreciated that second portion 18 can be the outer portion and first portion 16 can be the inner portion.

It will be appreciated that first portion 16 and second portion 18 also may have other configurations. For example, second portion 18 may be completely surrounded by first portion 16 or vice versa. Alternatively, rather than forming one strip bisecting first portion 16, second portion 18 may form two or more strips. Antiperspirant product 10 may also comprise a third portion and other additional portions that do not comprise the compositions of first portion 16 and second portion 18. First portion 16 and second portion 18 may take any other configuration suitable for applying the portions to skin. The surface 20 of first portion 16 and surface 22 of the second portion 18 each comprises at least 15%, and preferably at least 25%, of the application surface 14. Each surface 20 and 22 may even comprise, for example, at least 40% of the application surface 14.

The first portion is a water-in-oil emulsion comprising a water phase mixed with an oil phase. Preferably, the first portion comprises a water phase in an amount of about 35 to about 45 weight percent (wt. %) of the total first portion and an oil phase in an amount of about 55 to about 65 wt. % of the first portion. The first portion preferably has a soft, non-flowing, solid composition that can be rubbed or wiped across the skin, particularly the underarm. However, the various embodiments are not so limited and the first portion can also have a gel, cream, or lotion consistency. The solid composition is substantially snow white in color, thus suggesting a clean and/or sterile nature.

In one exemplary embodiment, the water phase of the first portion comprises a water-soluble active antiperspirant compound. Active antiperspirant compounds contain at least one active ingredient, typically metal salts, that are thought to reduce sweating by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so sweat can not diffuse to the skin surface. Some active antiperspirant compounds that may be used in the first portion include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x (H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zironyl hyroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the antiperspirant compound is aluminum sesquichlorohydrate (anhydrous) with glycine and calcium chloride. In another embodiment, the first portion comprises an active antiperspirant compound present in the amount of 0 to about 25 weight percent (USP). As used herein, weight percent (USP) or wt. % (USP) of an antiperspirant salt is calculated as anhydrous weight percent in accordance with the U.S.P. method, as is known in the art. This calculation excludes any bound water and glycine. In a more preferred embodiment, the antiperspirant compound comprises aluminum sesquichlorohydrate (anhydrous) and glycine and calcium chloride at an active level of about 12 to about 25 wt. % (USP).

In an exemplary embodiment, the water phase also comprises at least one water soluble carrier/solubilizer present in a sufficient amount to solubilize or disperse the water phase ingredients of the antiperspirant product. Such carriers/solubilizers suitable for use in the antiperspirant product include, but are not limited to, propylene glycol, glycerol, dipropyl glycol, ethylene glycol, butylene glycol, propylene carbonate, dimethyl isosorbide, hexylene glycol, ethanol, n-butyl alcohol, n-propyl alcohol, isopropyl alcohol, and the like. In a preferred embodiment, the water phase comprises propylene glycol and, in a more preferred embodiment, the water phase comprises propylene glycol in an amount of about 4 to about 12 wt. % of the total first portion. In addition to the carrier/solubilizer, the first portion comprises water. The water evaporates from the antiperspirant product upon application of the antiperspirant product to the skin, providing a cooling sensation to the skin.

The water phase also may comprise optional ingredients that serve a particular purpose. In one exemplary embodiment, the water phase comprises an activator for the active antiperspirant compound. In one embodiment, the water phase comprises calcium chloride and in another embodiment comprises calcium chloride in an amount of about 0.7 to about 2 wt. % of the total first portion.

The oil phase of the first portion comprises an emulsifier of cetyl PEG/PPG-10/1 dimethicone, in accordance with an exemplary embodiment. Cetyl PEG/PPG-10/1 dimethicone is a copolymer of cetyl dimethicone and an alkoxylated derivative of dimethicone containing an average of 10 moles of ethylene oxide and 1 mole of propylene oxide. The use of cetyl PEG/PPG-10/1 dimethicone as an emulsifier in the first portion causes the first portion to exhibit skin feel characteristics that are typical of deodorant products. For example, with cetyl PEG/PPG-10/1 dimethicone, the antiperspirant products glide onto the skin with less friction while still maintaining a solid consistency for easy application. In addition, when applied, the antiperspirant products are smoother than typical antiperspirants and thus give the skin a smoother and softer feel. In a preferred embodiment, the oil phase comprises cetyl PEG/PPG-10/1 dimethicone in an amount of about 1 to about 4 wt. % of the total antiperspirant product.

Further included in the oil phase of the first portion is at least one structurant and/or gellant (hereinafter referred collectively as structurant) that facilitates the solid consistency of the first portion. Naturally-occurring or synthetic waxy materials or combinations thereof can be used as such structurants. Examples of these waxy materials include those fatty alcohols that are solid at room temperature and hydrocarbon waxes or silicone waxes. Such materials are widely available, and by suitable selection of the materials themselves and their concentrations in the formulation, it is possible to obtain either a soft solid or a firm solid. In a preferred embodiment, the oil phase comprises a high molecular weight (MW) polyethylene. As used herein, the term “high molecular weight polyethylene” or “high MW polyethylene” means polyethylene having a molecular weight of 200 to 5000 daltons (Da). In a more preferred embodiment, the oil phase comprises high MW polyethylene having a molecular weight of about 500 Da. In another preferred embodiment, the oil phase comprises high MW polyethylene in an amount of about 5 to about 15 wt. % of the total first portion. In this regard, polyethylene can be used in smaller amounts as a structurant in the first portion than other structurants, such as stearyl alcohol. Stearyl alcohol is commonly used as a structurant in solid stick underarm products. However, stearyl alcohol has a tendency to leave visible white deposits on the skin, and the deposits can also transfer onto clothing when the clothing comes into contact with the skin. Accordingly, in another preferred embodiment, the oil phase comprises substantially no stearyl alcohol. The term “substantially no stearyl alcohol” as used herein means no stearyl alcohol or stearyl alcohol in an amount that is sufficiently small so that it would not cause visible white residue to deposit on skin and/or clothing after application of the antiperspirant product to the skin.

In accordance with another exemplary embodiment, when high MW polyethylene is used in the oil phase as a structurant, the oil phase also comprises at least one low MW synthetic wax. In addition to facilitating the high MW polyethylene by serving a structurant function, the low MW synthetic wax also improves the manufacturing processes of the antiperspirant products. Generally, polyethylene has a relatively high melting point (70-100° C.) and, thus, as described in more detail below, the oil phase of the first portion must be heated to this high melting point to melt the polyethylene. However, this high temperature heating may result in higher manufacturing costs and unpredictable and/or non-repeatable yields of the antiperspirant product. The presence of an effective amount of low MW synthetic wax (synthetic wax having a molecular weight in the range of 1200-2900 Da) modifies the high MW polyethylene, lowering the melting point of the polyethylene. In an exemplary embodiment, the low MW synthetic wax is present in the oil phase in an amount of about 0 to about 3 wt. % of the total first portion. In another exemplary embodiment, the low MW synthetic wax has a molecular weight of about 1800. In addition to improving hardness of the first portion, the low MW synthetic wax reduces syncresis and tackiness and also has a high refractive index (R.I.) that minimizes and/or prevents a white residue on the skin by masking the antiperspirant metallic salt(s) that stays upon the skin upon evaporation of the carrier(s), described in more detail below. As used herein, the term “high refractive index” means an refractive index no less than about 1.4.

The oil phase further comprises at least one hydrophobic carrier. An example of suitable hydrophobic carriers includes liquid siloxanes and particularly volatile polyorganosiloxanes, that is, liquid materials having a measurable vapor pressure at ambient conditions. The polyorganosiloxanes can be linear or cyclic or mixtures thereof. The linear volatile silicones generally have viscosities of less than about 5 centistokes at 25° C., while the cyclic volatile silicones have viscosities under 10 centistokes. Preferred siloxanes include cyclomethicones, which have from about 3 to about 6 silicon atoms, such as cyclotetramethicone, cyclopentamethicone, and cyclohexamethicone, and mixtures thereof. The carrier also may comprise, additionally or alternatively, nonvolatile silicones such as dimethicone and dimethicone copolyols, which have from about 2 to about 9 silicon atoms. Examples of suitable dimethicone and dimethicone copolyols include polyalkyl siloxanes, polyalkylaryl siloxanes, and polyether siloxane copolymers.

The oil phase may also comprise a high R.I. hydrophobic compound. The high R.I. hydrophobic compound minimizes and/or prevents a white residue on the skin by masking the antiperspirant metallic salt that stays upon the skin upon evaporation of the carrier(s). Examples of high R.I. hydrophobic compounds for use in the antiperspirant products include C₁₂-C₁₅ alkyl benzoate, such as Finsolv TN® available from Innospec of the United Kingdom, PPG-14 butyl ether, and phenyl trimethicone. In a preferred embodiment, the oil phase comprises C₁₂-C₁₅ alkyl benzoate and, in a more preferred embodiment, the oil phase comprises C₁₂-C₁₅ alkyl benzoate in an amount of about 2 to about 12 wt. % of the total first portion.

The first portion of the antiperspirant product contemplated herein also may comprise additives, such as those used in conventional antiperspirants. For example, in addition to, or instead of, antiperspirant efficacy, the first portion may comprise additives that cause the antiperspirant product to exhibit long-lasting fragrance, odor protection, bacteria control, and/or another desired purpose and/or function These additives include, but are not limited to, fragrances, including encapsulated fragrances, dyes, pigments, preservatives, antioxidants, moisturizers, and the like. These optional ingredients can be included in the first portion in an amount of 0 to about 20 wt. %.

The second portion of the antiperspirant product is a solid wax formula comprising an anhydrous, hydrophobic vehicle including a volatile silicone and/or high melting component. In one exemplary embodiment, the second portion further comprises an active antiperspirant compound suspended in the anhydrous, hydrophobic vehicle. Any of the active antiperspirant compounds listed above for use in the first portion may be used in the second portion. In one exemplary embodiment, the same active antiperspirant compound or compounds is used in both the first portion and the second portion. Alternatively, the second portion may comprise a different active antiperspirant compound or compounds than the first portion. If present, the second portion comprises the active antiperspirant compound in a perspiration-reducing effective amount. In one embodiment, the second portion comprises the active antiperspirant compound in an amount of about 0 to about 25 wt. % (USP). In a preferred embodiment, the second portion comprises the active antiperspirant compound in an amount of about 3 to about 25 wt. % (USP). More preferably, the second portion comprises the active antiperspirant compound in an amount of about 8 to about 22 wt. % (USP).

The high melting components may include any material suitable for use in an antiperspirant stick that melts at a temperature of about 70° C. or higher. Typical of such materials are the high melting point waxes. These include beeswax, spermaceti, carnauba, bayberry, candelilla, montan, ozokerite, ceresin, paraffin waxes, semi-microcrystalline and microcrystalline waxes, hydrogenated jojoba oil, and hydrogenated castor oil (castor wax). The preferred wax is hydrogenated castor oil. Other suitable high melting components include various types of high melting gelling agents such as polyethylene-vinyl acetate copolymers, polyethylene homopolymers, 12-hydroxystearic acid, and substituted and unsubstituted dibenzylidene alditols. Typically, the high melting components comprise about 1 to about 25 wt. %, preferably about 2 to about 15 wt. %, of the composition. Volatile silicones include cyclomethicones and dimethicones, discussed above.

Other components may include, for example, non-volatile silicones, polyhydric alcohols having 3-6 carbon atoms and 2-6 hydroxy groups, fatty alcohols having from 12 to 24 carbon atoms, fatty alcohol esters, fatty acid esters, fatty amides, non-volatile paraffinic hydrocarbons, polyethylene glycols, polypropylene glycols, polyethylene and/or polypropylene glycol ethers of C₄-C₂₀ alcohols, polyethylene and/or polypropylene glycol esters of fatty acids, and mixtures thereof. The term “fatty” is intended to include hydrocarbon chains of about 8 to 30 carbon atoms, preferably about 12 to 18 carbon atoms.

Non-volatile silicones include polyalkylsiloxanes, polyalkylaryl siloxanes, and polyethersiloxanes with viscosities of about 5 to about 100,000 centistokes at 25° C., polymethylphenylsiloxanes with viscosities of about 15 to about 65 centistokes, and polyoxyall kylene ether dimethylsiloxane copolymers with viscosities of about 1200 to about 1500 centistokes.

Useful polyhydric alcohols include propylene glycol, butylenes glycol, dipropylene glycol and hexylene glycol. Fatty alcohols include stearyl alcohol, cetyl alcohol, myristyl alcohol, oleyl alcohol, and lauryl alcohol. Fatty alcohol esters include C₁₂₋₁₅ alcohols benzoate, myristyl lactate, cetyl acetate, and myristyl octanoate. Fatty acid esters include isopropyl palmitate, myristyl myristate, and glyceryl monostearate. Fatty amides include stearamide MEA, stearamide MEA-stearate, lauramide DEA, and myristamide MIPA.

Non-volatile paraffinic hydrocarbons include mineral oils and branched chain hydrocarbons with about 16 to 68, preferably about 20 to 40, carbon atoms. Suitable polyethylene glycols and polypropylene glycols will typically have molecular weights of about 500 to 6000, such as PEG-10, PEG-40, PEG-150 and PPG-20, often added as rheology modifiers to alter product appearance or sensory attributes.

Polyethylene and/or polypropylene glycol ethers or C₄-C₂₀ alcohols include PPG-10 butanediol, PPG-14 butyl ether, PPG-5-buteth-7, PPG-3-isostearth-9, PPG-3-myreth-3, oleth-10, and steareth-20. Polyethylene and/or polypropylene glycol esters of fatty acids include PEG-8 distearate, PEG-10 dioleate, and PPG-26 oleate. These are generally added to give emollient properties.

The above list of materials is by way of example only and is not intended to be a comprehensive list of all potential components of the antiperspirant products contemplated herein. Other high and low melting waxes, volatile and non-volatile compounds and other suitable components are readily identifiable to those skilled in the art. Of course, other ingredients such as colloidal silicas, particulate polyolefins, talcum materials, colorants and preservatives may also be included as desired. For example, the composition may include up to about 10% fragrance or about 2% colorant by weight.

As noted above, in addition to, or instead of, an active antiperspirant compound, the first portion and/or the second portion may comprise a component or components that cause the first portion and/or the second portion to exhibit or impart a desired function or purpose in addition to, or instead of, antiperspirant efficacy. For example, the second portion may comprise deodorant active ingredients. A suitable deodorant active is any agent that inhibits, suppresses, masks or neutralizes malodor. These may include (1) antimicrobial or bactericidal agents that kill the bacteria responsible for malodor production, (2) agents that inhibit or suppress or interfere with the bacterial enzymatic pathway that produces malodor, and (3) agents that mask or absorb or neutralize malodor. “Fragrances” as used herein are not considered deodorant active ingredients. Examples of deodorant actives include triclosan, triclocarban, usnic acid salts, zinc phenolsulfonate, b-chloro-D-alanine, D-cycloserine, animooxyacetic acid, cyclodextrine, and sodium bicarbonate. Alternatively, or in addition, the portions may comprise fragrances, for example, in an amount that imparts a long-lasting fragrance to the antiperspirant product.

In accordance with exemplary embodiments, a method for manufacturing the antiperspirant product illustrated in FIGS. 1 and 2 is shown in FIGS. 3-19. Using this method, the antiperspirant product is molded directly within the container, using the container as a mold for the antiperspirant product, and delivering fluid (molten) antiperspirant product to the container to form an antiperspirant composition 25 illustrated in FIG. 3. It will be appreciated, however, that the invention is not limited to use of the container as a mold and that any satisfactory mold may be used for manufacturing the antiperspirant product. Referring to FIG. 3, container 12 has an application end 24 and an opposite end 26. The container 12 also contains a factory seal 28, which is placed over the application surface 14 of antiperspirant product 10 to protect it during shipment and to render it tamper-proof prior to purchase, and a cover 30. The factory seal 28 is removed by the user, and the cover is used during storage of the product between uses. As the product is exhausted, it is advanced from the container by the user using advancement device 32, e.g., a screw mechanism as shown, at opposite end 26 of container 12.

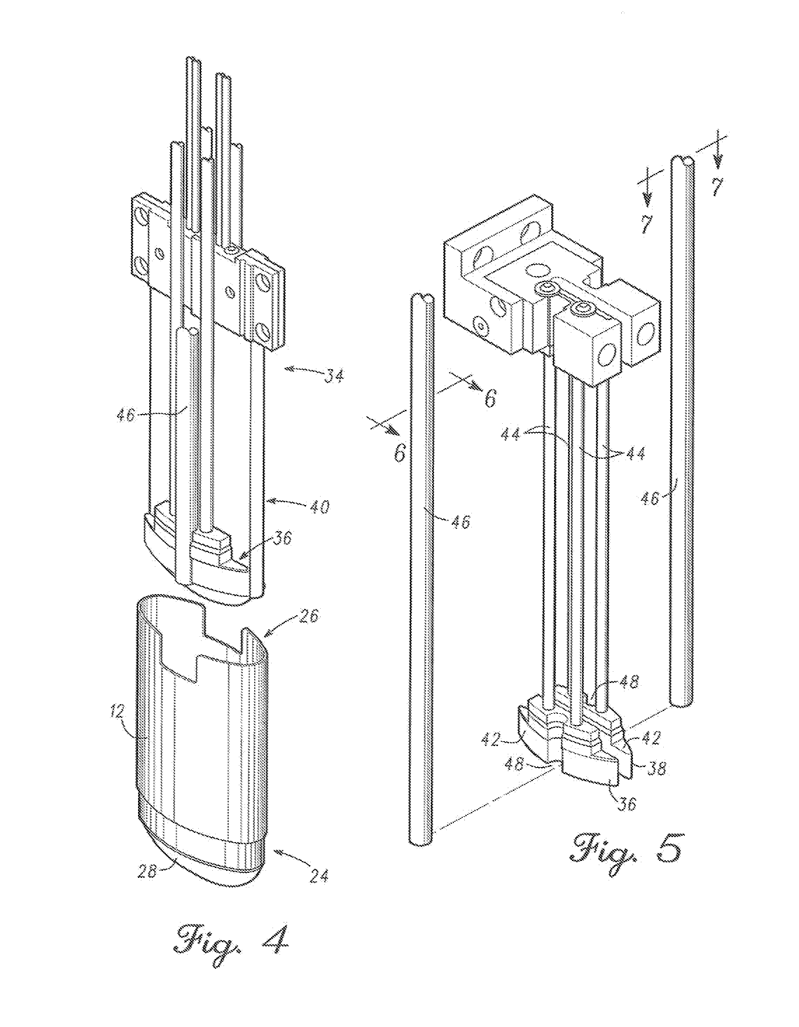

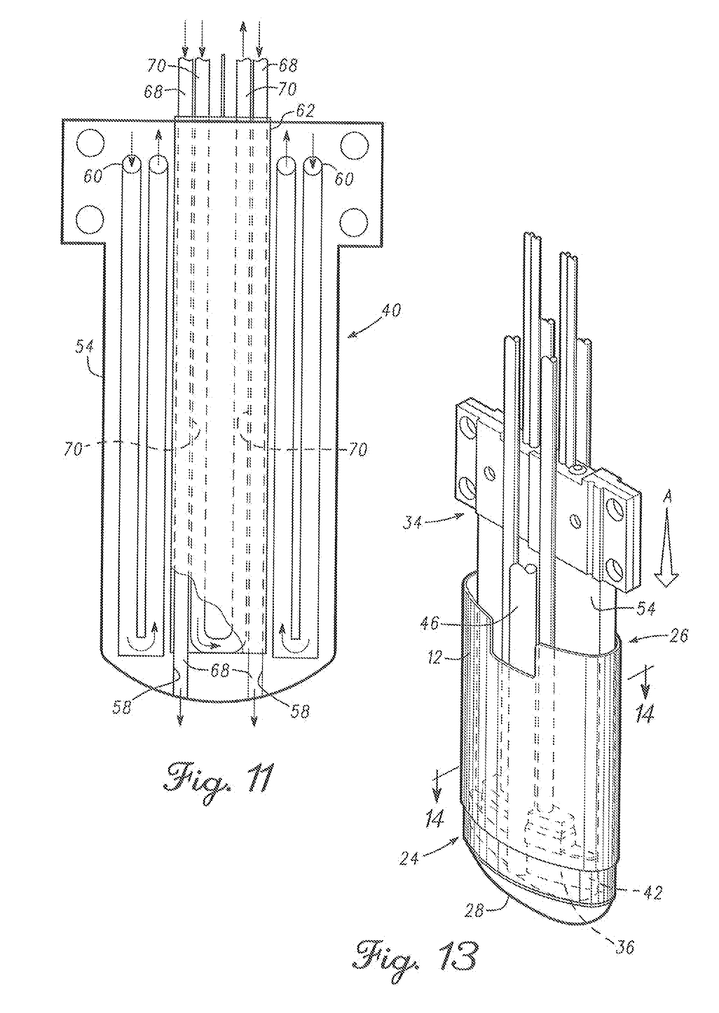

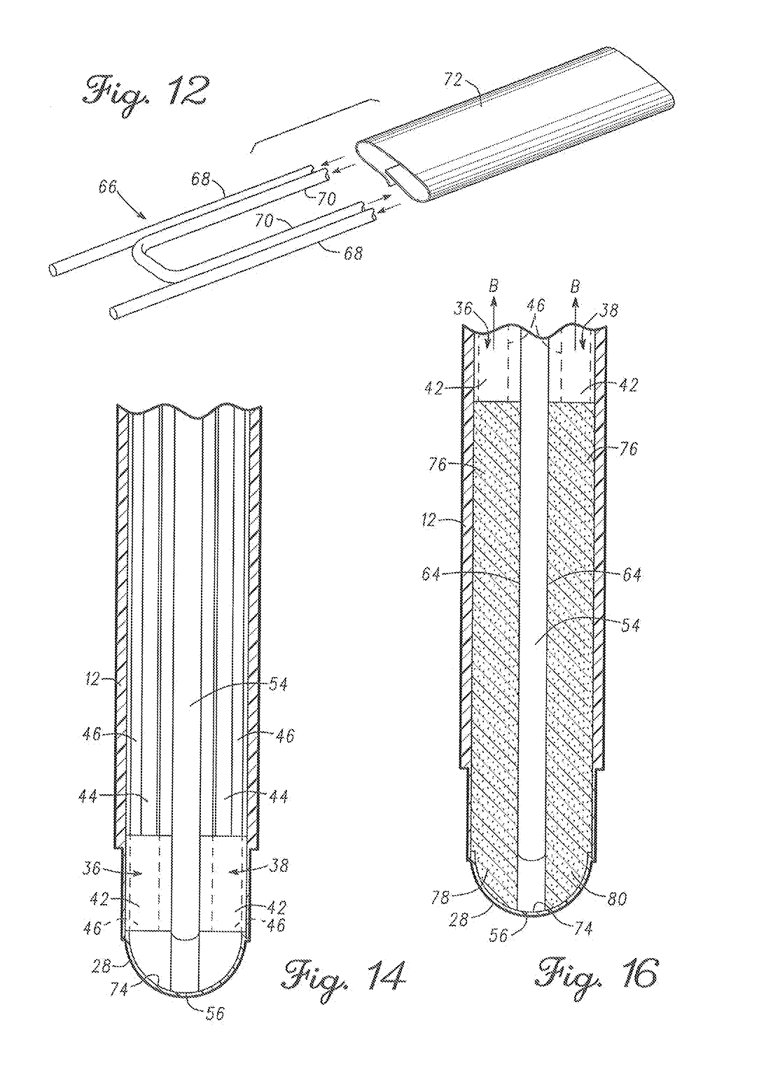

Referring to FIG. 4, a filling assembly 34 is positioned above opposite end 26 of an empty container 12. The factory seal 28 is in place, sealing the application end 24 of the container 12. The filling assembly 34 is lowered into the container 12 and is used to fill two compositions into the container, as will be described below with reference to FIGS. 13-18. The filling assembly 34 will first be described herein.

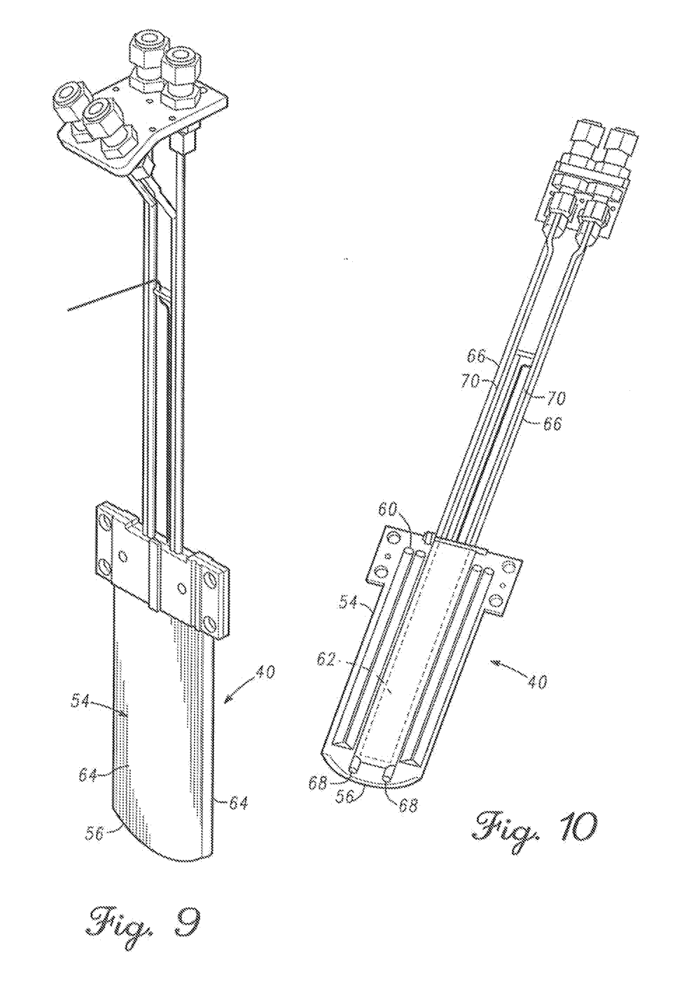

The components of filling assembly 34 are shown individually and in detail in FIGS. 5-10. The filling assembly consists of two outer nozzle assemblies 36 and 38, as shown in FIG. 5, and an inner nozzle assembly 40, as shown in FIG. 9. A first portion of the antiperspirant product is delivered by the outer nozzle assemblies 36 and 38, and a second portion is delivered by the inner nozzle assembly 40. The term “nozzle” as used herein refers to any device that is capable of delivering a fluid composition.

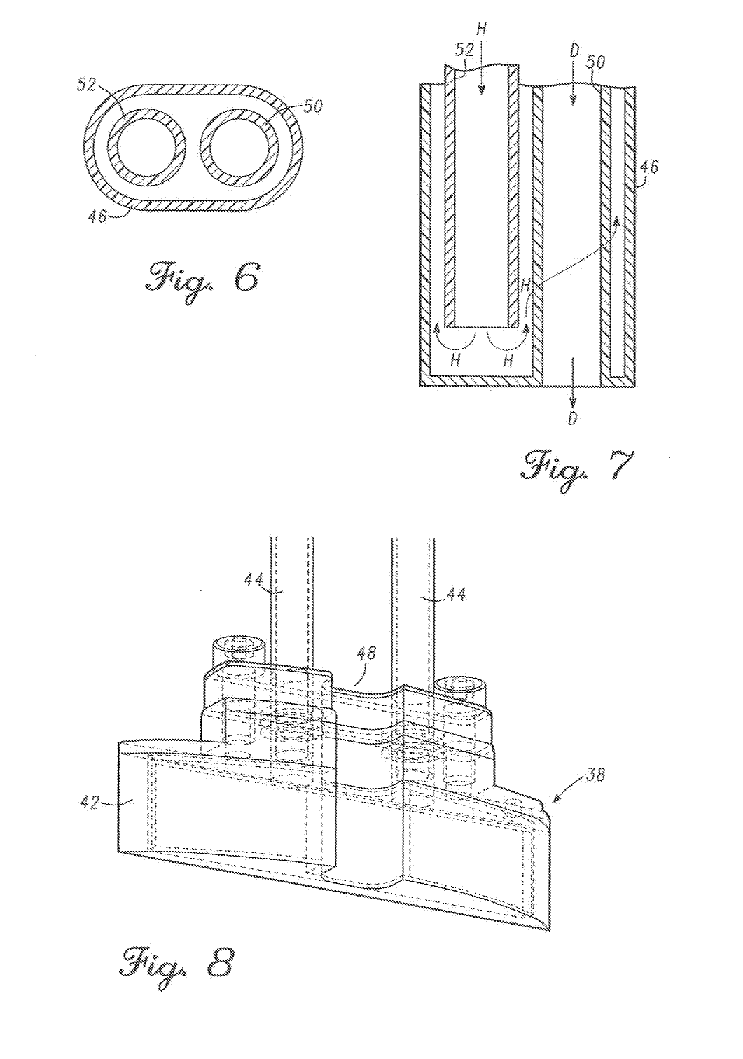

Each of the outer nozzle assemblies 36 and 38, one of which is shown in detail in FIG. 8, include a scraper body 42 that is mounted on two outer tubes 44. The scraper body is hollow, and is chilled by the circulation of cooling media. Its function will be discussed below. A center nozzle tube 46, disposed between the outer tubes 44, is retained in a groove 48 (FIG. 8) in the scraper body (center nozzle tube 46 is omitted in FIG. 8 for clarity). The two outer tubes 44 support the scraper body 42, allow it to be moved vertically, and circulate cooling media to and from the scraper body. As shown in FIGS. 6 and 7, the center nozzle tube 46 consists of a delivery tube 50 and a heating tube 52. The heating tube circulates heating media (arrows H, FIG. 7) to maintain the first portion in a molten state as it is delivered thought the delivery tube 50 (arrows D, FIG. 7). A temperature sensor, e.g., a thermocouple, thermistor, or the like (not shown), may he provided on one or both of the scraper bodies to measure the temperature of the scraper body.

Inner nozzle assembly 40 includes a housing 54 that provides a molding surface for the first portion, as will be discussed below. Housing 54 includes a curved leading edge 56 shaped to sealingly engage the inner surface of the factory seal 28. If desired, leading edge 56 may be a relatively sharp edge to provide a concentrated pressure against the factory seal 28. The interior of housing 54 is shown in FIGS. 10, 11, and 12. The housing 54 defines a pair of delivery channels 58, a pair of substantially U-shaped cooling channels 60, and a central rectangular channel 62. The cooling channels circulate cooling media to chill the outer surface 64 of housing 54. The central rectangular channel 62 receives an assembly 66, shown in FIG. 12, that includes a pair of delivery tubes 68 brazed to a substantially U-shaped heating tube 70. Heating tube 70 circulates heating media to heat the second phase as it is being delivered through the delivery tubes 68. The assembly 66 is wrapped in insulation 72 (FIG. 12) to insulate it within the chilled housing. The inner nozzle assembly 40 may also include one or more temperature sensors (not shown) to determine the heating and/or cooling temperatures.

The process of filling the container 12, and thus molding the antiperspirant product 10, is shown in FIGS. 13-18. First, as shown in FIGS. 13 and 14, the filling assembly 34 is inserted into the container 12, through opposite end 26 (arrow A), until leading edge 56 of the housing 54 contacts the inner surface 74 of factory seal 28. At this stage of the process, both the inner nozzle assembly 40 and the outer nozzle assemblies 36 and 38 arc fully extended into the container 12. Although the cover 30 is omitted in FIGS. 4 and 13-18, for clarity, the cover is in place during the molding process. Cover 30 provides a flat surface on which the container can rest during filling, and also holds the factory seal in place against the downward pressure exerted by the inner nozzle assembly.

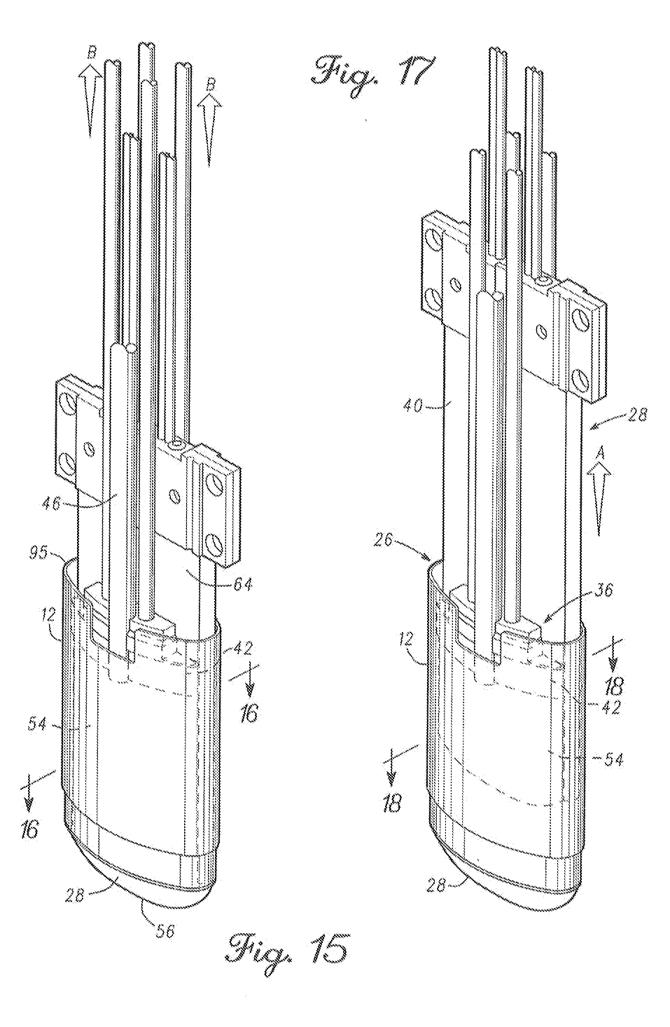

Next, as shown in FIGS. 15 and 16, a first portion 76, in liquid form (herein “the first fluid portion”), of the antiperspirant product is delivered to the container 12 to the open spaces on both sides of housing 54. For purposes of example, the first fluid portion herein will be an emulsion antiperspirant product as described above. Accordingly, to deliver the first fluid portion to the container in liquid form, the first fluid portion can be maintained at a temperature in the range of about 75 to about 80° C., for example, about 75° C. The :first fluid portion 76 is delivered from delivery tubes 50 of outer nozzle assemblies 36 and 38, while the outer nozzle assemblies 36 and 38 are being simultaneously raised, as indicated by arrows B. During delivery of the first fluid portion, the inner nozzle assembly 40 is maintained in its lowered position so that leading edge 56 provides a seal against the inner surface 74 of factory seal 28 to prevent first fluid portion 76 from flowing under the leading edge 56 and so that the outer surface 64 of housing 54 provides a molding surface. Sealing is provided by the curved surface of leading edge 56, which corresponds closely to the curvature of surface 74 of factory seal 28. Sealing can be enhanced by applying downward pressure to the inner nozzle assembly 40 during delivery of the first portion 16. The first fluid portion is molten, so that it is sufficiently fluid for delivery, but will solidify quickly as it cools. Because the outer surface 64 is chilled, the first fluid portion 76 will solidify relatively quickly.

The container 12, filled with the first fluid portion 76, is shown in FIG. 16. After the container 12 has been filled to a desired level, the first fluid portion 76 is allowed to solidify sufficiently so that a skin or thin solid layer will form to prevent the first fluid portion 76 from mixing with a second fluid portion. A skin thickness of from about 1 to about 2 millimeters (mm) is generally sufficient, typically requiring a dwell time of from about 1 to about 10 seconds, preferably from about 2 to about 6 seconds at about −10 to +20° C. The skin will form adjacent surface 64 of housing 54 due to the chilling of surface 64. The dwell time will depend on the temperature to which the surface 64 of housing 54 is cooled, and the temperature of the molten material when it is delivered. The resulting two regions 78 and 80 of the first fluid portion 76 (FIG. 12) will define the regions having the surfaces 20 of first portion 16 (FIG. 1).

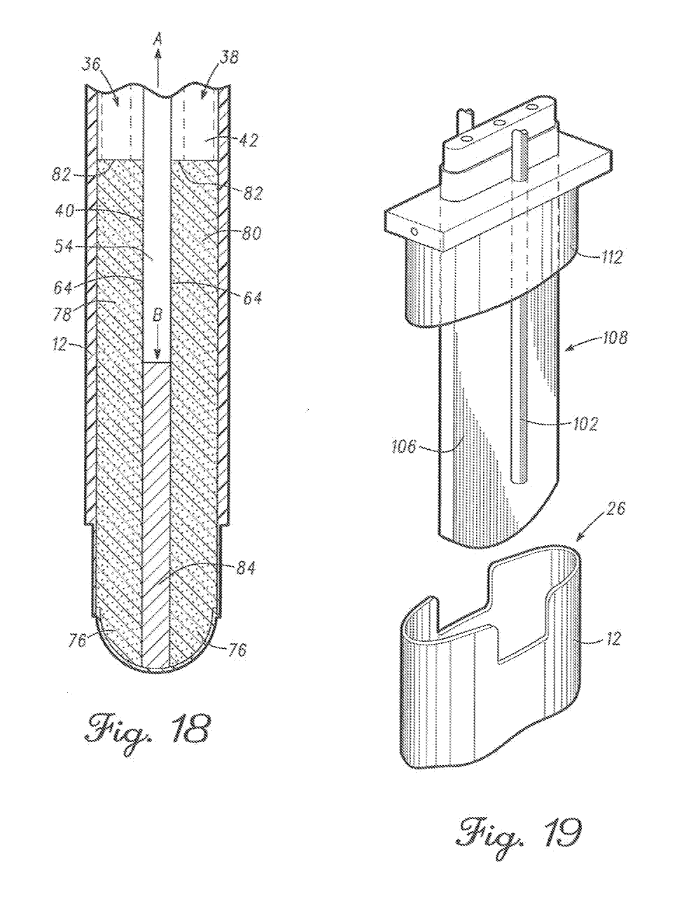

During this dwell time, and then during the subsequent filling step described below, outer nozzle assemblies 36 and 38 are maintained in a position, shown in FIG. 18, at which a lower surface 81 of each scraper body 42 is in contact with the top surface of the regions 78 and 80 of first fluid portion 76. In this position, the chilled scraper bodies serve several functions: (a) they aid in solidification of the top surface of the first fluid portion 76, (b) they hold the first fluid portion 76 in the container during the next step, described below, and (c) they scrape the outer surface 64 of housing 54 during the next step, which helps the solidified skin to release from surface 64. With respect to the first function, the chilling of the top surface of the first fluid portion 76 causes a skin to form at the top surface, which extends from the inner wall of the container and thus provides lateral support to the regions 78 and 80, preventing them from collapsing or leaning inward. If additional lateral support is desired, the upward movement of the outer nozzle assemblies during the filling process can be interrupted, e.g., for about one second, at an intermediate position within the container. The intermediate position may be about halfway up. This brief pause in the filling operation will allow a skin to form under the scraper bodies 42 at this point, providing additional lateral support to the regions 78 and 80.

Referring to FIGS. 17 and 18, when the first fluid portion 76 has sufficiently solidified (formed a skin), the inner nozzle assembly 40 is moved upwards (arrow A), out of the container. As the inner nozzle assembly 40 moves upward, a second fluid portion 84 is delivered to the space that becomes available between regions 78 and 80 of the first fluid portion 76 as the housing 54 is removed, as indicated by arrow B in FIG. 18. The second fluid portion 84 is delivered through delivery channels 58 of the inner nozzle assembly 40. The second fluid portion will define second portion 18 of the finished product (FIG. 1).

The retraction of inner nozzle assembly 40 is coordinated with the delivery of second fluid portion 84 so that the volume vacated by the nozzle is immediately filled with the liquid volume that is being delivered. This prevents damage to the weak skin that supports region 78 and 80 and prevents intermingling of the fluid portions. This coordination may be achieved, e.g., by electronically linking servo motors that control a delivery pump to a screw that retracts the nozzle assembly.

During delivery of second fluid portion 84, the chilled scraper bodies prevent regions 78 and 80 from being lifted upwards by friction exerted by outer surface 64 of housing 54, help the skin to release from surface 64, and scrape off any of the first fluid portion 76 that adheres to outer surface 64 during removal of housing 54. This leaves the outer surface 64 of housing 54 clean prior to filling of a new container.

The steps shown in FIGS. 17 and 18 complete the molding process and the manufacturing of the antiperspirant composition 25 illustrated in FIG. 3. Solidification of the first and second fluid portions is completed by cooling the product, for example, by passing the filled container through a forced air tunnel operating at between about 10 to about 25° C. The finished product (FIG. 3) is completed by sealing the open opposite end 26 with a package base (not shown) that includes advancement device 32.

Suitable materials for housing 54 include metals such as stainless steel, aluminum alloys, copper or beryllium. Coated metals can also be used, e.g., stainless steel coated with titanium nitride, chromium, or electroless nickel with a polytetrafluoroethylene (PTFE) infusion; aluminum coated with aluminum oxide hardcoat anodizing, hardcoat anodizing with a PTFE infusion, or electroless nickel with or without a PTFE infusion; or aluminum plated with nickel or chrome. The housing may be coated with a release coating such as liquid silicone to enhance release of the skin.

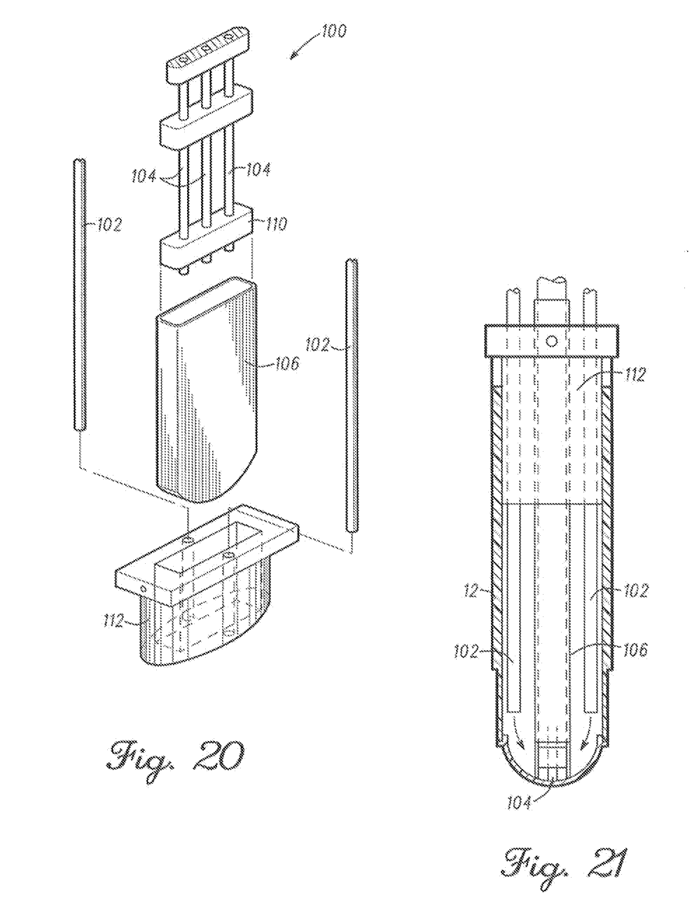

An alternative molding process can be performed using the filling assembly 108 shown in FIGS. 19 and 20. In this embodiment, inner nozzle assembly 100 includes a plurality of delivery tubes 104 (FIG. 20) surrounded by a housing 106 that can be raised and lowered independently of delivery tubes 104. The outer nozzle assemblies discussed above are replaced by outer nozzles 102 and scraper block 112, with scraper block 112 performing the functions described above with reference to the scraper bodies 42. (If desired, the outer nozzle assemblies discussed above may be used in this embodiment.)

Because, in this embodiment, the housing 106 can be moved independently of the delivery tubes 104, the first and second fluid portions can be filled in any desired order, or simultaneously. If they are filled simultaneously, as shown in FIG. 21, the housing 106 would be left in place for a sufficient length of time to allow at least one of the compositions to form a skin to prevent intermingling of the two fluid portions. Thus, the housing 106 may be moved upward slowly, a few seconds behind the nozzles.

In this embodiment, it is generally useful that the inside surface of the housing 106 be scraped. To accomplish this, member 110 (FIG. 20) is mounted on the delivery tubes 104, and shaped to closely lit the interior of housing 106 so that, when the delivery tubes 104 are moved vertically relative to the housing 106, member 110 will scrape the inner surface of the housing.

The following is an exemplary embodiment of an antiperspirant product, with each of the components set forth in weight percent of the antiperspirant product. The example is provided for illustration purposes only and is not meant to limit the various embodiments of the antiperspirant product in any way. All materials are set forth in weight percent.

### Example 1

### General

First Portion

| | Wt. % |
|---|---|
| Water Phase -Ingredient | |
| Water | 20-50 |
| Aluminum Sesquichlorohydrate | 12-25 |
| and glycine and calcium chloride | |
| Propylene Glycol | 4-12 |
| Oil Phase -Ingredient | |
| Cyclomethicone | 18-25 |
| C₁₂₋₁₅ Alkyl Benzoate | 2-12 |
| Cetyl PEG/PPG-10/1 Dimethicone | 1-4 |
| Polyethylene | 5-15 |
| Synthetic Wax | 0-3 |
| Fragrance | 0-2 |
| Total | 100 |

Second Portion

| Ingredient | Wt. % |
|---|---|
| Cyclomethicone | qs |
| Aluminum Sesquichlorohydrate | 0-25 |
| and glycine and calcium chloride | |
| Stearyl Alcohol | 15-20 |
| PPG-14 Butyl Ether | 8-11 |
| Hydrogenated Castor Oil | 2-6 |
| Myristyl Myristate | 1-3 |
| Fragrance | 1-7 |
| Silica Dimethyl Silylate | 0-1 |
| Silica | 0-0.5 |
| Pigment | 0-0.1 |
| Total | 100.0 |

FIRST PORTION EXAMPLES

| Ingredient | EX. 2 | EX. 3 | EX. 4 | EX. 5 |
|---|---|---|---|---|
| Water | 30.9994 | 47.3100 | 49.6000 | 30.3500 |
| AP Salt | 17.6000 | 17.6000 | 0.0000 | 21.7975 |
| Propylene Glycol | 8.0000 | 8.0000 | 8.0000 | 8.0000 |
| Cyclomethicone | 14.3106 | 0.0000 | 18.1500 | 18.1025 |
| C₁₂₋₁₅ Alkyl Benzoate | 8.0000 | 8.0000 | 8.0000 | 8.0000 |
| Cetyl PEG/PPG-10/ | 1.2500 | 1.2500 | 1.2500 | 1.2500 |
| 1-Dimethicone | | | | |
| Polyethylene | 10.5000 | 10.5000 | 11.0000 | 10.5000 |
| Synthetic Wax | 0.1000 | 0.1000 | 0.1000 | 0.1000 |
| Fragrance | 1.9000 | 1.9000 | 1.9000 | 1.9000 |
| Calcium Chloride | 3.1824 | 3.1824 | 0.0000 | 0.0000 |
| Glycine | 2.1576 | 2.1576 | 0.0000 | 0.0000 |
| Dimethicone | 2.0000 | 0.0000 | 2.0000 | 0.0000 |
| Total | 100.0000 | 100.0000 | 100.0000 | 100.0000, |

SECOND PORTION EXAMPLES

| Ingredient | EX. 2 | EX. 3 | EX. 4 |
|---|---|---|---|
| Cyclomethicone | 42.9398 | 0.0000 | 62.6250 |
| AP Salt | 21.6852 | 18.6630 | 0.0000 |
| Stearyl Alcohol | 17.0000 | 20.0000 | 17.0000 |
| PPG-14 Butyl Ether | 9.0000 | 10.0000 | 9.0000 |
| Hydrogenated Castor Oil | 2.8400 | 3.5000 | 2.8400 |
| Myristyl Myristate | 1.9200 | 0.0000 | 1.9200 |
| Fragrance | 3.9000 | 3.9000 | 3.9000 |
| Silica Dimethyl Silylate | 0.5600 | 0.0000 | 0.5600 |
| Silica | 0.1400 | 1.1434 | 0.1400 |
| Pigment | 0.0150 | 0.0150 | 0.0150 |
| C₁₃₋₁₆ Isoparaffin, C₁₂₋₁₄ | 0.0000 | 42.7786 | 0.0000 |
| Isoparaffin, C₁₃₋₁₅ Alkane | | | |
| Cetyl Alcohol | 0.0000 | 0.0000 | 2.0000 |
| Total | 100.0000 | 100.0000 | 100.0000 |

Accordingly, antiperspirant products that exhibit antiperspirant efficacy and that also provide a smooth glide when applied to the skin have been described. The various embodiments of the antiperspirant products comprise a first portion and a second portion. The first portion is a solid emulsion that comprises cetyl PEG/PPG-10/1 dimethicone as an emulsifier. The use of cetyl PEG/PPG-10/1 dimethicone as an emulsifier in an antiperspirant product causes the antiperspirant product to glide onto skin with less friction, that is, in a smoother manner, than conventional antiperspirants while still maintaining a solid consistency for easy application. The second portion is an invisible solid wax formula that provides enhanced antiperspirant efficacy, long-lasting :fragrance, odor protection, bacteria control, and/or another desired purposes and/or functions. The first portion, the second portion, or both portions of the antiperspirant product can provide antiperspirant efficacy. Upon application, the two portions combine to provide a user with an antiperspirant product that exhibits a dry, non-tacky skin feel without crumbling or caking of the product on the skin.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant product comprising:
a container; and
an antiperspirant product housed within the container, the antiperspirant product comprising:
a first portion comprising an emulsion having a water phase and an oil phase, wherein the oil phase comprises cetyl PEG/PPG-10/1 dimethicone; and
a second portion having a composition different from the first portion.

2. The antiperspirant product of claim 1, wherein the first portion comprises a first region and a second region and the second portion is positioned between the first region and the second region.

3. The antiperspirant product of claim 1, wherein the first portion comprises an active antiperspirant compound.

4. The antiperspirant product of claim 3, wherein the first portion comprises the active antiperspirant compound in an amount of about 12 to about 25 wt. % (USP).

5. The antiperspirant product of claim 3, wherein the second portion comprises an active antiperspirant compound.

6. The antiperspirant product of claim 5, wherein the first portion and the second portion comprise the same active antiperspirant compound.

7. The antiperspirant product of claim 5, wherein the second portion comprises the active antiperspirant compound in an amount of about 3 to about 25 wt. % (USP).

8. The antiperspirant product of claim 3, wherein the active antiperspirant compound is aluminum sesquichlorohydrate (anhydrous) with glycine and calcium chloride.

9. The antiperspirant product of claim 1, wherein the second portion comprises an active antiperspirant compound.

10. The antiperspirant product of claim 1, wherein the cetyl PEG/PPG-10/1 dimethicone is present in the oil phase in an amount of 1 to about 4 wt. % of the first portion.

11. The antiperspirant product of claim 1, wherein the water phase is present in an amount of about 35 to about 45 wt. % of the first portion.

12. The antiperspirant product of claim 1, wherein the oil phase is present in an amount of about 55 to about 65 wt. % of the first portion.

13. An antiperspirant product comprising:
an outer portion comprising an emulsion having a water phase and an oil phase and wherein the oil phase comprises cetyl PEG/PPG-10/1 dimethicone; and
an inner portion that at least substantially bisects the outer portion, the inner portion having a composition different from the outer portion.

14. The antiperspirant product of claim 13, wherein the outer portion comprises an active antiperspirant compound.

15. The antiperspirant product of claim 14, wherein the inner portion comprises an active antiperspirant compound.

16. The antiperspirant product of claim 15, wherein the inner portion and the outer portion comprise the same active antiperspirant compound.

17. The antiperspirant product of claim 14, wherein the active antiperspirant compound is aluminum sesquichlorohydrate (anhydrous) with glycine and calcium chloride.

18. The antiperspirant product of claim 13, wherein the inner portion comprises an active antiperspirant compound.

19. The antiperspirant product of claim 13, wherein the cetyl PEG/PPG-10/1 dimethicone is present in the oil phase in an amount of 1 to about 4 wt. % of the outer portion.

20. The antiperspirant product of claim 13, wherein the water phase is present in an amount of about 35 to about 45 wt. % of the outer portion.

21. The antiperspirant product of claim 13, wherein the oil phase is present in an amount of about 55 to about 65 wt. % of the outer portion.

22. A method for manufacturing an antiperspirant product, the method comprising the steps of:
depositing a first portion of the antiperspirant product into a mold, wherein the first portion comprises an emulsion having a water phase and an oil phase and wherein the oil phase comprises cetyl PEG/PPG-10/1 dimethicone;
allowing the first portion to at least partially solidify;
depositing a second portion of the antiperspirant product into the mold, wherein the second portion has a composition different from the first portion; and
allowing the second portion to at least partially solidify.

23. The method of claim 22, wherein the step of depositing a first portion of the antiperspirant product into a mold comprises depositing the first portion into a container that will be used to store and dispense the antiperspirant product.

24. The method of claim 22, wherein the step of depositing the second portion is performed before the step of depositing the first portion.

25. The method of claim 22, wherein the step of depositing a first portion comprises depositing the first portion comprising an active antiperspirant compound.

26. The method of claim 25, wherein the step of depositing a second portion comprises depositing the second portion comprising an active antiperspirant compound.

27. The method of claim 26, wherein the steps of depositing comprise depositing the first portion comprising an active antiperspirant compound and depositing the second portion comprising the same active antiperspirant compound.

28. The method of claim 25, wherein the step of depositing a first portion comprises depositing the first portion comprising aluminum sesquichlorohydrate (anhydrous) with glycine and calcium chloride.

29. The method of claim 22, wherein the step of depositing a second portion comprises depositing the second portion comprising an active antiperspirant compound.

30. The method of claim 22, wherein the step of depositing a first portion comprises depositing the first portion having the cetyl PEG/PPG-10/1 dimethicone present in the oil phase in an amount of 1 to about 4 wt. % of the second portion.

31. The method of claim 22, wherein the step of depositing a first portion comprises depositing the first portion having the water phase present in an amount of about 35 to about 45 wt. % of the first portion.

32. The method of claim 22, wherein the step of depositing a first portion comprises depositing the first portion having the oil phase is present in an amount of about 55 to about 65 wt. % of the first portion.
